(19) United States
(12) Patent Application Publication
(10) Pub. No.: US 2004/0049009 A1
(43) Pub. Date: Mar. 11, 2004

(54) Methods for improving the antagonistic/agonistic properties of peptidic antagonists/agonists of the corticotropin-releasing factor receptor (crfr)

(76) Inventors: Klaus Eckart, Freidland (DE); Joachim Spiess, Gottingen (DE); Olaf Jahn, Gottingen (DE)

Correspondence Address: BIRCH STEWART KOLASCH & BIRCH, PO BOX 747, FALLS CHURCH, VA, 22040-0747, US

(21) Appl. No.: 10/380,446

(22) PCT Filed: Sep. 24, 2001
(86) PCT No.: PCT/EP01/11031

(30) Foreign Application Priority Data
Sep. 22, 2000 (DE) 100 47 186.2
Nov. 3, 2000 (DE) 100 54 539.4
Sep. 20, 2001 (EP) 01122446.6

(51) Int. Cl. C07K 14/705; G06G 7/48; G06G 7/58; C12P 21/06

(52) U.S. Cl. 530/350; 435/69.1; 703/11

(57) ABSTRACT

The present invention relates to a method for improving the antagonistic/agonistic properties of peptidic antagonists/agonists of the corticotropin-releasing factor receptor (CRFR). Further, the present invention relates to an antagonist of the ligand of the corticotropin-releasing factor receptor (CRFR) comprising or alternatively consisting of the amino acid sequence of astressin wherein at least Ala at position 11 is replaced by another amino acid. Further, the present invention relates to an antibody directed against the agonist or antagonist of the present invention. Also described is an anti-idiotypic antibody which is directed against the antibody(ies) of the invention. The present invention also relates to a pharmaceutical or diagnostic composition comprising the antagonist, the agonist, the antibody(ies) and/or the anti-idiotypic antibody of the invention. Furthermore, the present invention relates to a kit comprising the agonist, the antagonist, the antibody(ies) and/or the anti-idiotypic antibody of the present invention. Also described is the use of the agonist, the antagonists, the antibody(ies) and/or the anti-idiotypic antibody of the invention for the preparation of a pharmaceutical composition for the treatment, diagnosis and/or prevention of corticotropin-releasing factor receptor-associated diseases. The present invention also relates to a method of refining the agonist and/or the antagonists of the present invention by means of peptidomimetics and synthesizing the refined compound. Furthermore, the present invention relates to a method of formulating the agonist/antagonist of the invention into a pharmaceutical composition.

The present invention relates to a method for improving the antagonistic/agonistic properties of peptidic antagonists/agonists of the corticotropin-releasing factor receptor (CRFR). Further, the present invention relates to an antagonist of the ligand of the corticotropin-releasing factor receptor (CRFR) comprising or alternatively consisting of the amino acid sequence of astressin wherein at least Ala at position 11 is replaced by another amino acid. Further, the present invention relates to an antibody directed against the agonist or antagonist of the present invention. Also described is an anti-idiotypic antibody which is directed against the antibody(ies) ” of the invention. The present invention also relates to a pharmaceutical or diagnostic composition comprising the antagonist, the agonist, the antibody(ies) and/or the anti-idiotypic antibody of the invention. Furthermore, the present invention relates to a kit comprising the agonist, the antagonist, the antibody(ies) and/or the anti-idiotypic antibody of the present invention. Also described is the use of the agonist, the antagonists, the antibody(ies) and/or the anti-idiotypic antibody of the invention for the preparation of a pharmaceutical composition for the treatment, diagnosis and/or prevention of corticotropin-releasing factor receptor-associated diseases. The present invention also relates to a method of refining the agonist and/or the antagonists of the present invention by means of peptidomimetics and synthesizing the refined compound. Furthermore, the present invention relates to a method of formulating the agonist/antagonist of the invention into a pharmaceutical composition.

Several documents are cited throughout the text of this specification either by name or are referred to by numerals to within parenthesis. Each of the documents cited herein (including any manufacturer's specifications, instructions, etc.) are hereby incorporated herein by reference; however, there is no admission that any document cited is indeed prior art as to the present invention.

Corticotropin-releasing factor (CRF), believed to synchronize the endocrine, autonomic, immunologic and behavioral responses to stress, was characterized as a 41-residue polypeptide (Spiess, J., J. Rivier, C. Rivier, and W. Vale, Proc. Natl. Acad. Sci. USA 78:6517-6521, 1981) on the basis of its ability to stimulate the secretion of adrenocorticotropic hormone (ACTH) from the anterior pituitary (Vale, W., J. Spiess, C. Rivier, and J. Rivier, Science 213:1394-1397,1981).

CRF exhibits its activity through G protein-coupled receptors. CRF receptor, type 1 (CRFR1), mainly found in pituitary and brain was cloned from human, mouse, rat, chicken, and frog (Vita, N., P. Laurent, S. Lefort, P. Chalon, J.-M. Lelias, M. Kaghad, G. Le Fur, D. Caput, and P. Ferrara, FEBS Lett. 335:1-5,1993; Chen, R., K. A. Lewis, M. H. Perrin, and W. Vale, Proc. Natl. Acad. Sci. USA 90:8967-8971, 1993; Perrin, M. H., C. J. Donaldson, R. Chen, K. A. Lewis, and W. Vale, Endocrinology 133:3058-3061, 1993; Chang, C.-P., R. V. Pearse I I, S. O'Connell, and M. G. Rosenfeld, Neuron 11:1187-1195, 1993; Yu, J., L. Y. Xie, and A. B. Abou-Samra, Endocrinology 137:192-197, 1996; Dautzenberg, F. M., K. Dietrich, M. R. Palchaudhuri, and J. Spiess, J. Neurochem. 69:1640-1649, 1997). cDNAs coding for two splice variants of CRF receptor, type 2, CRFR2α and CRFR2β, were cloned from brain, heart, and skeletal muscle (Lovenberg, T. W., C. W. Liaw, D. E. Grigoriadis, W. Clevenger, D. T. Chalmers, E. B. De Souza, and T. Oltersdorf, Proc. Natl. Acad. Sci. USA 92:836-840, 1995; Perrin, M., C. Donaldson, R. Chen, A. Blount, T. Berggren, L. Bilezikjian, P. Sawchenko, and W. Vale, Proc. Natl. Acad. Sci. USA 92:2969-2973, 1995; Kishimoto, T., R. V. Pearse I I, C. R. Lin, and M. G. Rosenfeld, Proc. Natl. Acad. Sci. USA 92:1108-1112, 1995; Stenzel, P., R. Kesterson, W. Yeung, R. D. Cone, M. B. Rittenberg, and M. P. Stenzel-Poore, Mol. Endocrinol. 9:637-645, 1995). In rodents, CRFR2a has been exclusively found in the central nervous system (CNS), whereas CRFR2β is predominantly distributed in the periphery. In humans, both receptor subtypes have been found in the CNS (Valdenaire, O., T. Giller, V. Breu, J. Gottowik, and G. Kilpatrick, Biochim. Biophys. Acta 1352: 129-132,1997). Recently, it has been proposed that urocortin (Ucn), a natural CRF analog, is the endogenous ligand to CRFR2 (Vaughan, J., C. Donaldson, J. Bittencourt, M. H. Perrin, K. Lewis, S. Sutton, R. Chan, A. V. Turnbull, D. Lovejoy, C. Rivier, J. Rivier, P. E. Sawchenko, and W. Vale, Nature (London) 378:287-292, 1995).

In addition, CRF binds to a soluble CRF binding protein (CRFBP). Present evidence indicates that the physiological role of endogenous CRFBP is to complex ligand, thereby reducing CRF receptor signaling. CRFBP is capable of binding human/rat CRF (h/rCRF) with high affinity. In contrast, other members of the CRF peptide family such as sauvagine (Svg) and ovine CRF (oCRF) are bound with affinities that are lower by two or three orders of magnitude, respectively. Substitution of residues 22, 23 and 24 of oCRF by their h/rCRF counterparts identified the ARAE motif as the decisive stretch of amino acid residues for high affinity binding of h/rCRF to CRFBP (S. W. Sutton et al., Endocrinology 136: 1097-1102; 1995).

CRF is assumed to play a major role in a number of neuropsychiatric diseases including affective disorders, anxiety disorders, anorexia nervosa and Alzheimer's disease (Behan, D. P., S. C. Heinrichs, J. C. Troncoso, X. J. Liu, C. H. Kawas, N. Ling, and E. B. De Souza, Nature (London) 378:284-287,1995). Furthermore, CRF modulates in vivo central effects such as memory and learning, food intake, locomotor activity, and anxiety. Some of these effects can be blocked by local injection of the peptidic antagonist astressin (Ast). Recent developments of peptidic CRF antagonists led to the N-terminally truncated peptides α-helical CRF⁹⁻⁴¹ (α-hel-CRF⁹⁻⁴¹), the cyclic CRF analog astressin (Ast), which is composed of amino acid residues 12-41 of h/rCRF, and the (CRFR2) selective antagonist anti-sauvagine-30 (aSvg-30). α-hel-CRF⁹⁻⁴¹ and Ast do not distinguish between CRFR1 and CRFR2. Furthermore, α-hel-CRF⁹⁻⁴¹ functions as a partial agonist. Ast could not be used for icv injection because of its limited solubility. This may be explained by the neutral pl of 7.5 for Ast obtained by pl calculation. Furthermore, Ast showed a moderate affinity to the rat CRF binding protein (rCRFBP) and therefore the effective concentration in vivo may be reduced by binding to CRFBP.

Thus, the technical problem underlying the present invention was to provide methods for improving the antagonistic/agonistic properties of peptidic CRF-antagonists/-agonists.

The solution to said technical problem is provided by the embodiments characterized in the claims.

Accordingly, the present invention relates to a method for improving the antagonistic/agonistic properties of peptidic antagonists/agonists of the corticotropin-releasing factor receptor (CRFR) comprising the steps of aligning the amino acid sequences of at least two antagonists/agonists of the corticotropin-releasing factor receptor (CRFR) which differ in their antagonistic/agonistic properties; identifying at least one position wherein the amino acid sequences are different; exchanging or replacing at least one amino acid which is different in the aligned amino acid sequences; and comparing the difference in the antagonistic/agonistic properties of the antagonists/agonists which comprise at least one exchanged or replaced amino acid and thereby identifying at least one amino acid which is responsible for said difference. Thus, the obtained antagonists/agonists which comprise at least one exchanged or replaced amino acid may also be seen as derivatives of the antagonist/agonist which have been known in the art.

Studies which have been carried out with h/rCRF and ovine CRF (oCRF) revealed no differences with respect to the affinity and in vitro potency on CRFR1, whereas the affinity to CRFBP of oCRF is lower by three orders of magnitude compared with h/rCRF. This difference exclusively depends on the exchange of the amino acid residues 22, 23, and 25, which represent the ARAE motif. Sauvagine (Svg), which is more hydrophilic than oCRF and h/rCRF, differs in the amino acid residues 21, 22, and 23 with respect to the ARAE motif from h/rCRF and binds with the same affinity as h/r CRF to CRFR1 with higher affinity to CRFR2, but with a significantly lower affinity than h/rCRF to rCRFBP. The two affinities differ by two orders of magnitude (Jahn, O. et al (2001), Peptides 22, 47-56).

Theoretical consideration about the physicochemical properties of the exchanged amino acids and the results known from comparison of oCRF and h/rCRF pointed to the position 22 of h/rCRF and the corresponding position 21 of Svg as the amino acid controlling the high affinity binding to CRFBP. On this basis a Svg derivative was synthesized by replacing the Glu²¹ of Svg by the corresponding residue Ala²² of h/rCRF and the inverted h/rCRF analog by exchange Ala²² to Glu. The altered properties of the obtained derivatives [A²¹]SVG and [E²²]h/rCRF were analysed as indicated in the appended examples. Surprisingly, it could be demonstrated that the replacement of Glu²¹ in Svg by Ala increased the affinity to CRFBP by two orders of magnitude. The opposite effect was observed when Ala²² in h/rCRF was exchanged correspondingly to obtain [E²²]h/rCRF. It is therefore concluded that position 22 of the ARAE motif in h/rCRF is a crucial hydrophobic contact point controlling the affinity to CRFBP. In contrast, [A²¹]SVG and [E²²]h/rCRF did not display significantly different affinities to CRFR1 or CRFR2 and [E²²]h/rCRF exhibited a slightly increased affinity to CRFR1 or CRFR2 when compared with the natural peptides Svg and h/rCRF.

Since the proposed physiological role of CRFBP is to function as a CRF trap, it can be argued that a CRF receptor antagonist with low affinity to CRFBP would be an antagonist having improved antagonistic properties within the meaning of the present invention. Thus, the introduction of Glu at the position corresponding to 22 in h/rCRF represents a useful tool to selectively switch off the affinity of an antagonist to CRFBP. Furthermore, this exchange is also advantageous in view of the hydrophilicity of the respective peptide and improves its solubility under physiologic conditions e.g at neutral pH. The peptides carrying Ala in the respective position showed indistinguishable high affinity of about 1 nM to CRFBP, whereas the analogs containing Glu in this respective position showed indistinguishable low affinity of about 100 nM to rCRFBP. In addition and most surprisingly the affinity to CRFR1 and CRFR2 was not significantly altered by the amino acid exchange in h/rCRF and Svg. Thus, it could be demonstrated that the method of the invention can advantageously be used to improve the antagonistic/agonistic properties of an “natural” antagonist of CFR which was already known in the art. In the context of the present invention the term “antagonist” denotes a peptidic antagonist which reduces or prevents the interaction of the CFR-receptor with its ligand/s. This can, e.g., be effected by blocking its binding site in order to reduce and or inhibit the binding of CRF. Test-systems which are suitable to test for such antagonistic properties are well-known in the art and, further, are shown in the appended examples. The term “ligand” which is used in the context of the present invention encompasses any molecule capable of specifically binding to the Corticotropin-Releasing Factor Receptor(s), including, e.g., (the) naturally occurring, endogenous ligand(s) of CRFR, or any compound(s) recombinantly or chemically synthesized or biochemically modified and capable of binding and activating CRFR. Further, a peptidic antagonist in the context of the present invention relates to a CRF-like peptide which binds with high affinity to CRFR and does not activate CRFR as e.g. determined by the low levels of CAMP produced after treatment with 1 μM antagonist relative to the levels produced by stimulation with 10 nM h/rCRFor 10 nM Svg of a CRFR1 or CRFR2 producing cell, respectively. Preferably, such an antagonist comprises the N-terminal truncation of the CRF-like peptide down to the C-terminal 30 amino acid residues including the G-terminal amidation.

In contrast an agonist in the context of the present invention relates to a CRF-like peptide which is able to activate CRFR as e.g. determined by the similar levels of CAMP produced after stimulation with an agonist concentration of 10×EC50 relative to the levels produced by stimulation with 10 nM h/rCRF or 10 nM Svg of a CRFR1 or CRFR2 producing cell, respectively. Preferably, such an agonist comprises the C-terminal 38 amino acid residues including the C-terminal amidation as depicted in the figures.

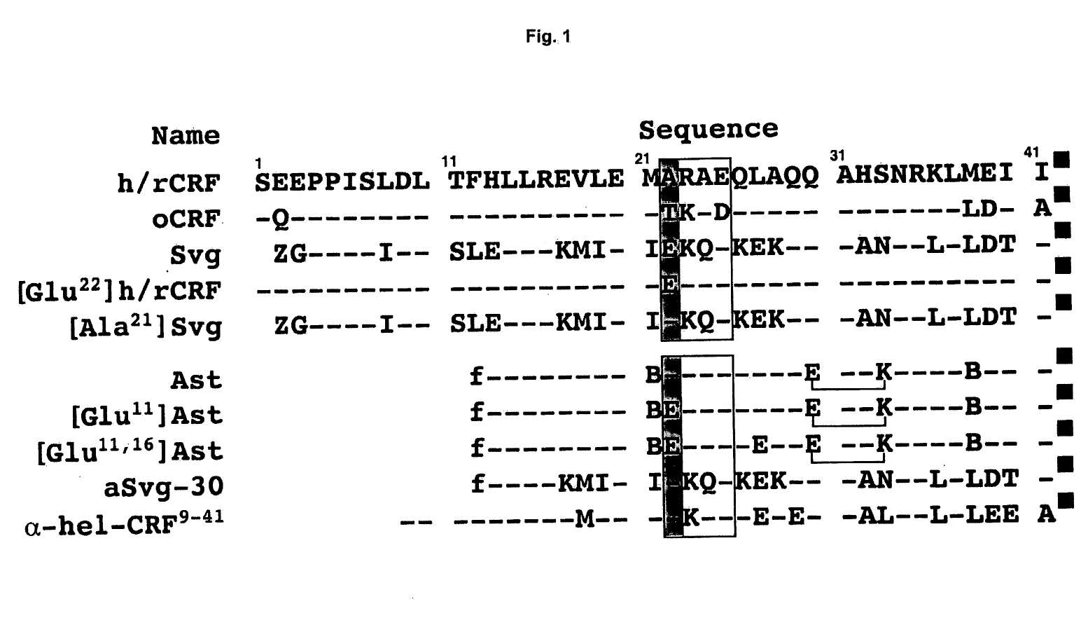

The term “aligning” in the context of the present invention relates to the alignment of at least two amino acid sequences of two members of peptidic antagonists/agonists of the corticotropin-releasing factor receptor (CRFR) by methods known in the art, e.g. by means of computer aided analysis via suitable computer-programs like BLAST which is, inter alia, available via http://www.ncbi.nim.nih.gov/BLAST or other computer-programs and/or methods which are known in the art (it is, for example, possible to sort the sequences beginning with the C-terminal amino acid one upon another without the incorporation of gaps in order to identify the respective position(s) as depicted in FIG. 1).

However, it is also envisaged that at least three, at least four, at least five and/or at least six amino acid sequences of two members of peptidic antagonists/agonists of the corticotropin-releasing factor receptor (CRFR) are aligned by computer aided analysis or other well-known methods for the alignment of peptidic sequences.

In accordance with the above, the peptidic CRF-antagonists/agonists of the present invention are derivatives of antagonist/agonists which are already known in the art i.e. whose antagonistic or agonistic properties with respect to CRF, CRFBP and/or the respective CRF-receptors is known. However, also peptidic antagonist/agonist of the CRFR are encompassed by the present invention which are not yet known or whose antagonistic/agonistic properties are not yet known, since it will be appreciated by the person skilled in the art that the underlying peptide sequence as well as the antagonistic/agonistic properties of such not yet known peptidic antagonist/agonist can be determined by methods well known in the art and/or by methods indicated in the appended examples. The term “derivative” in this context relates to peptidic antagonists/agonists wherein at least one amino acid in the sequence of said peptidic antagonists/agonists is replaced or exchanged. Of course it is also envisaged that more than one amino acid can be replaced in the sequence of the derivatives of the antagonists/agonists of the invention as long as these changes in the sequence improve the antagonistic/agonistic properties of said antagonists/agonists. Thus, it is envisaged that the derivatives of the antagonists/agonists of the present invention comprise at least two, at least three, at least four, at least five, at least six, at least seven, at least eight, at least nine and/or at least ten exchanged and or replaced amino acids. However, the derivatives of the antagonists/agonists of the present invention may comprise even more exchanged and or replaced amino acids in their sequence as long as these changes in the sequence improve the antagonistic/agonistic properties of said antagonists/agonists. It will be appreciated by the person skilled in the art that the exchanged or replaced amino acids within the peptidic antagonists/agonists of the invention comprise single amino acids or stretches of more than one amino acids which are exchanged together or a combination thereof. Furthermore, the term “derivatives” also encompasses further modification(s) of the peptidic antagonist/agonist which was improved by the methods of the invention like, e.g. labeling of the peptides or modification(s) of the peptides as indicated herein.

It is envisaged that the antagonists/agonists of the present invention have improved antagonistic/agonistic properties when compared with their “natural” counterparts i.e. the peptidic antagonists/agonists of CRFR which are already known in the art. Accordingly, the term “natural” as mentioned herein relates to those antagonists/agonists of CRFR which have not been improved by the methods of the present invention. However, it will be appreciated that the term “natural” as mentioned herein does not necessarily mean that the such antagonists/agonists of CRFR which have not been improved by the methods of the present invention are derived from a biological source, although they may be derived from such a biological source.

Accordingly, the meaning of the term “antagonists with improved antagonistic properties” in accordance with the present invention relates to peptidic antagonists which have been improved by the methods of the present invention and thereby have at least one of the following features:

i. they have an increased solubility at neutral pH

ii. they have a decreased affinity to CRFBP; and

iii. the affinity ratio to the CRFR is not changed or, alternatively, the affinity ratio to the CRFR is increased.

Accordingly, the meaning of the term “agonist with improved agonistic properties” in the meaning of the present invention relates to agonists which have been improved by the methods of the present invention and subsequently have at least one of the following features:

i they have an increased solubility at neutral pH;

ii they have an increased affinity to CRFBP;

iii the affinity to the CRFR is not significantly increased; and

iv the EC₅₀ for receptor activation is not altered or decreased, wherein the term “EC₅₀” in this context relates to the concentration of ligand at 50% of its maximum biological response.

The term “neutral pH” in the context of the present invention relates to a preferred pH range of pH 7,25 to pH 7,55, a more preferred pH-range from pH 7,3 to pH 7,5, and to an even more preferred pH-range from pH 7,35 to pH 7,45. It is most preferred that the neutral pH in context of the present invention is pH 7,4.

Thus, in accordance with the present invention, the term “improving the antagonistic/agonistic properties” relates to a peptidic antagonist/agonist which has been improved by the methods of the present invention, wherein at least one of the antagonistic/agonistic properties as mentioned herein is improved in comparison to the natural antagonist/agonist from which the improved peptidic antagonist/agonist of the invention is derived from. In a preferred embodiment the improved antagonist/agonist of the invention is improved in all of the above mentioned properties, e.g. it is most preferred that an improved peptidic antagonist of the invention has (i) increased solubility at neutral pH as described herein, (ii) has a decreased affinity to CRFBP and (iii) the affinity ratio to the CRFR is not changed or, alternatively, the affinity ratio to the CRFR is increased. Accordingly, it is most preferred that an improved peptidic agonist of the present invention has (i) increased solubility at neutral pH as described herein, (ii) has a decreased affinity to CRFBP (iii)-the affinity to the CRFR1 is not significantly decreased and (iv) the EC₅₀ for receptor activation is not decreased, wherein the term “EC₅₀” in this context relates to the concentration of ligand at 50% of its maximum biological response.

The term ‘increased solubility’ in this context refers to the increased concentration at room temperature of a saturated peptide solution in artificial cerebrospinal fluid (aCSF). The concentration of the peptide in a saturated solution is performed by quantitative amino acid analysis. The term “increased solubility” as used in accordance with the present invention relates to the increased concentration at room temperature of a saturated peptide solution in artificial cerebrospinal fluid (aCSF) of more than 10% more than 20%, more than 30% more than 40% more than 60% more than 80%. It is most preferred that the increased concentration at room temperature of a saturated peptide solution in artificial cerebrospinal fluid is more than 100%. The solubility of the antagonistic/agonistic peptides of the invention can be measured as indicated in the appended examples. Accordingly, the term “decreased solubility” in this context refers to the decreased concentration at room temperature of a saturated peptide solution. Further, the term “increased affinity refers to a decreased IC₅₀ value determined in a competition experiment using a radioactive labelled competitor as described herein. Accordingly, the term “decreased affinity” refers to an increased IC₅₀ value determined in a competition experiment using a radioactive labeled competitor i.e. by methods known in the art or as described herein. The term “not significantly increased” relates to changes within the 95% confidence intervals. Accordingly, the term “not significantly decreased” relates to changes within the 95% confidence intervals. Different amino acid stretches of CRF were analyzed which resemble different functions of said molecule (Beyermann et al (2000), J. Biol. Chem 275, 5702-5709). For example amino acid residues 12 to 20 and 31 to 41 are involved in binding to the CRFR wherein amino acid residues 21 to 30 resemble a helical connecting element. Furthermore, amino acid residues 4 to 11 are involved in the activation process of G-proteins and CRFR.

The term “peptidic” in the context of the present invention relates to a compound which is composed of amino acid residues, comprising and/or consisting of D- as well as L-amino acid residues or a combination thereof. Thus, it is also envisaged that the peptidic antagonists/agonists of the invention have a length of approximately 5 to 30 amino acid residues. Yet it is also envisaged that the peptidic antagonists/agonists of the invention have a length of about 10 to 40, 10 to 50, 10 to 60 and/or 10 to 70 amino acid residues although the length is not limited thereto. Yet it is also envisaged that the peptidic antagonists/agonists of the invention have a length of 5 to 15, 5 to 20, 5 to 25, 5 to 30, 5 to 35, 5 to 45 and/or 5 to 50 amino acid residues but the respective length is of course not limited thereto. The person skilled in the art will appreciate it that the peptidic antagonists/agonists of the invention can of course also have a length which differs from the lengths given above which is dependent from the respective length of the underlying natural antagonist/agonist which was improved by use of the methods of the invention to obtain derivatives of said antagonists/agonists which have improved antagonistic/agonistic properties. Thus, a suitable minimal length of the peptidic antagonists/agonists of the invention is about 5 amino acid residues in length and a suitable maximal length of the peptidic antagonists/agonists of the invention is about 60 amino acid residues in length. Furthermore, it is envisaged that the term “peptidic” also encompasses modifications of the antagonistic/agonistic peptides of the invention as described herein.

When it became clear that a single amino acid residue could switch the high affinity to CRFBP on or off without affecting the affinity to CRFR1 and CRFR2 we used this strategy to the natural CRFR antagonist Ast. The pl value of Ast is calculated with 7.5 whereas replacement of Ala²² by Glu decreased the calculated pi to 5.1. Therefore, [E¹¹]Ast was synthesized and tested for solubility, affinity to CRFBP, CRFR1, CRFR2, and the antagonistic potential on both receptor subtypes. With [E¹¹]Ast we obtained a concentration of 126 μM in aCSF at pH 7.4. Furthermore, [E¹¹]Ast was not able to compete with [¹²⁵I]Tyr⁰-h/rCRF for binding to CRFBP when applied in concentrations up to 3 μM, whereas Ast exhibited moderate affinity to CRFBP (IC₅₀=90 nM). In contrast, no changes in the affinity to CRFR1 compared with Ast were found. Also, Ast (9.8%) and [E¹¹]Ast (8.4%) did not differ significantly in their residual relative intrinsic activity as indicated by cAMP accumulation after application of 1 μM antagonist to HEK 293 cells expressing either CRFR1 or CRFR2. [E¹]Ast peptides inhibited to the same extent as Ast the accumulation of cAMP of transfected HEK cells producing CRFR1 or CRFR2 after application of 1 nM h/rCRF or 1 nM Svg, respectively. Thus, [E¹¹]Ast is a new CRFR antagonist which combines good solubility at pH 7.4 with no detectable affinity to CRFBP and full antagonistic properties on CRFR1 and CRFR2.

[E¹¹]Ast exhibited to both CRFR subtypes an affinity increased by one order of magnitude compared with Ast. [E¹¹]Ast showed indistinguishable intrinsic activity and relative potency on both receptor subtypes. No competition with radiolabeled h/rCRF for rCRFBP up to 3 μM could be detected. The maximum concentration of [E”]Ast at pH 7.4 was found to be 126 μM. As expected the hydrophobicity compared with Ast was significantly reduced. As determined by IEF measurements, the new antagonist [E¹¹]Ast was less charged at physiological pH than Ast. Thus, it was concluded, that in contrast to the initial expectation the solubility of Ast and [E¹¹]Ast was depending to a greater extent on the hydrophobicity rather than the charging of the peptide. This may be different at significantly higher or lower pH and with other peptides.

Thus, in a further embodiment of the method of the invention said method further comprises a step of replacing the amino acid identified by the methods of the invention in a further peptidic antagonist/agonist of the corticotropin-releasing factor receptor (CRFR). Accordingly, it is envisaged that at least one amino acid residue which was identified by the methods of the present invention is replaced or exchanged at the respective position in a sequence of another peptidic agonist/antagonist of CRFR. In this context it will be appreciated that said “other” peptidic antagonist/agonist represents a natural antagonist/agonist or an peptidic antagonist/agonist which was already improved by the methods of the present invention.

It is also a subject of the present invention to further improve the antagonistic/agonistic properties of an peptidic agonist/antagonist of CRFR by repeating the steps of the inventive methods which are mentioned herein. Accordingly, it is envisaged that e.g. in a first round of improvement of the antagonistic/agonistic properties of the above mentioned peptidic antagonists/agonists e.g. the solubility is increased, wherein in a, second round of improvement the binding affinity to the CRFBP is increased or decreased and so on.

In another embodiment the methods of the invention further comprise the step of refining the obtained antagonist/agonist of the invention by means of peptidomimetics comprising (a) modeling said antagonist/agonist by molecular modeling (i.e. biosym program) and (b) chemically synthesizing the modeled antagonist/agonist. Thus, it is envisaged that the antagonist/agonist of the present invention can be chemically synthesized according to methods well known in the art, e.g., solid phase synthesis with Fmoc or t-boc chemistry (see also, e.g., Rühmann, A., A. K. E. Köpke, F. M. Dautzenberg, and J. Spiess, Proc. Natl. Acad. Sci. USA 93:10609-10613, 1996).

In a preferred embodiment said peptidic antagonist/agonist of CRFR which is suitable for improvement by use of the methods of the present invention is selected from the group consisting of CRF, astressin, sauvagine, urotensin 1, urocortin, and urocortin like peptide.

In another preferred embodiment the present invention relates to an antagonist/agonist obtainable by the methods of the present invention. In a most preferred embodiment said antagonists of the invention are selected from a group consisting of [Glu”]Ast, [Glu^{11,16}]Ast. In another most preferred embodiment said agonists of the invention are selected from a group consisting of [A²¹]Svg, [A^{21,23}R²²]Svg and [E²²]h/rCRF.

In another preferred embodiment said antagonist is astressin. The cyclic CRF analog astressin (Ast) is composed of amino acid residues 12-41 of h/rCRF. The sequence of astressin is depicted below and also shown in FIG. 1.

In a further preferred embodiment, the present invention relates to a derivative of astressin comprising or alternatively consisting of the amino acid sequence Phe¹-His²-Leu³-Leu⁴-Arg⁵-Glu⁶-Val⁷-Leu⁸-Glu⁹-norleucine¹⁰-Ala¹¹-Arg¹²-Ala¹³-Glu¹⁴-Gln¹⁵-Leu¹⁶-Ala¹⁷-Gln¹⁸-Glu¹⁹-Ala²⁰-His²¹-Lys²²-Asn²³-Arg²⁴-Lys²⁵-Leu²⁶-norleucine²⁷-Glu²⁸-Ile²⁹-Ile³⁰-NH₂, wherein Glu¹⁹, (i.e. glutamic acid at position 19) and Lys²² (i.e. lysine at position 22 of the above depicted sequence) are connected via a lactam-bridge, wherein at least Ala¹¹, is replaced by another amino acid. In this respect, it is envisaged that at least positions 19 and 22 as indicated in the sequence of Astressin are not exchanged/replaced. Further it is envisaged that the Astressin-derivatives of the invention have a C-terminal amidation and that the N-terminal, amino acid residue is a D-amino acid residue.

In accordance with the present invention, the respective position of an amino acid within the above depicted amino acid sequence of an antagonist/agonist of the present invention is indicated via the small number exponent which appears next to the three-letter-code or single-letter code of the respective amino acid residue. Thus, e.g. “His²” relates to amino acid residue His which is located at position 2 in the sequence of the respective antagonist/agonist. Alternatively, the respective amino acid residue is indicated via the respective one-letter code which is also well known in the art. In this respect, e.g. the term “A¹¹” relates to the amino acid residue alanine at position 11 of the respective amino acid sequence. Accordingly it is envisaged, that the above mentioned three- and one-letter code of the respective amino acid residues are exchangeable without altering the meaning. For example, the term as exemplified as “[Glu^{11,16}]Ast” relates to an astressin derivative which has at positions 11 and 16 amino acid residue glutamine in its sequence. Alternatively, in line with the definition as given herein, e.g. the term “[Glu^{11,16}]Ast” is equally exchangeable with the term “[E^{11,16}]Ast” or “[Glul^{11,16}]Astressin” or “(E^{11,16}]Astressin”. The mentioned one-letter and three-letter code of the amino acid residues is, for example, described in Stryer, Biochemistry. Furthermore it will be appreciated by the person skilled in the art that the respective numbering of the “position” of an amino acid residue in the context of the present invention is based on the orientation of the underlying peptidic sequence, starting with the N-terminus. Accordingly, position one (1) denotes the N-terminal amino acid residue and so on.

The lactam bridge can be prepared, for example, as described recently (Rühmann et al., Proc. Natl. Acad. Sci. (1998) 95, 15264-15269) and as described in the appended examples.

In a preferred embodiment at least Ala¹¹ (i.e. the amino acid alanine at position 11 of the above depicted sequence of astressin) is replaced by an acidic and/or charged amino acid residue. In a preferred embodiment said amino acid is selected from the group consisting of amino acid residues Glu, Leu, Met, Gin, Lys, Arg, His, Thr, Ser, Ile, Phe and Asp.

In a more preferred embodiment the amino acid Leucine at position 16 of the Astressin-sequence as indicated above is replaced by an acidic and/or charged amino acid residue. In a preferred embodiment said amino acid is selected from the group consisting of amino acid residues Glu, Ala, Met, Gin, Lys, Arg, His, Thr, Ser, Ile, Phe and Asp.

In a most preferred embodiment amino acid Alanine at position 11 of the astressin sequence as depicted above is replaced by the amino acid residue Glutamine.

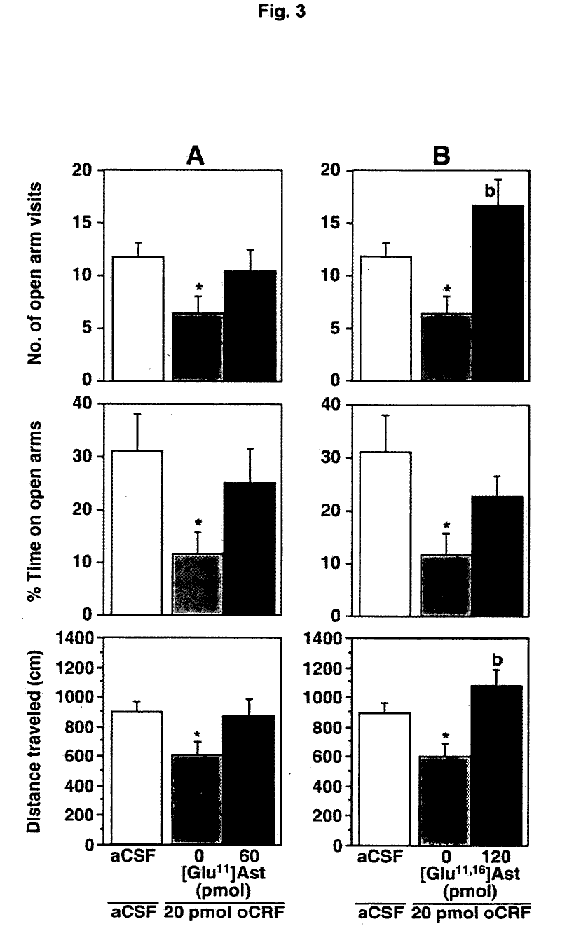

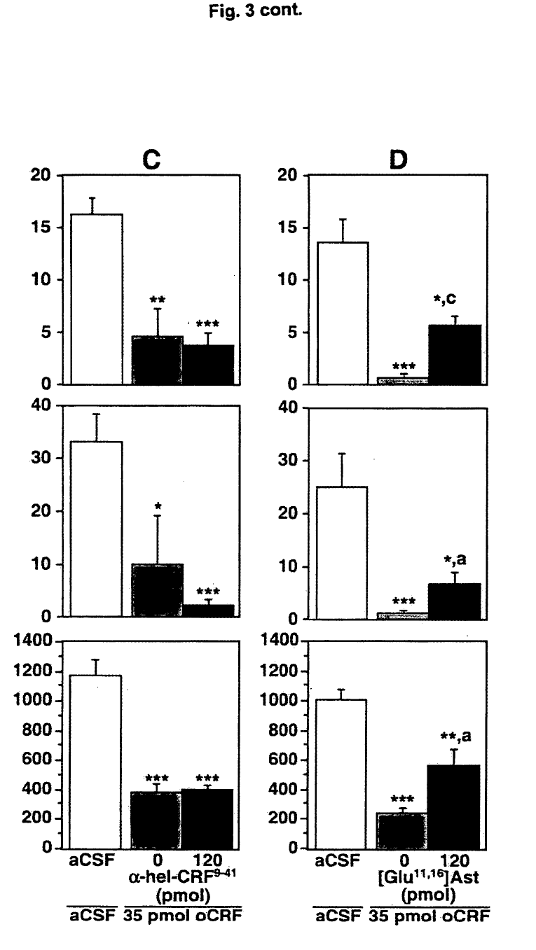

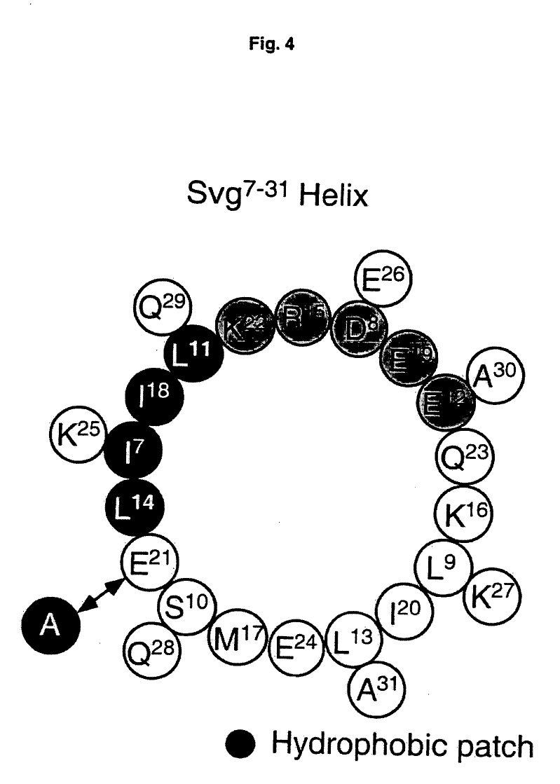

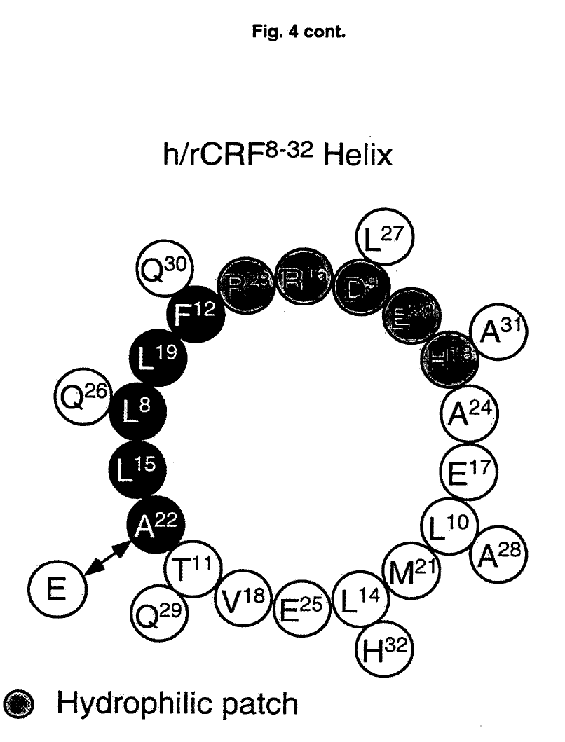

It was observed that Ast preinjected i.c.v. did not prevent oCRF-induced changes of the mice's behavior in the elevated plus-maze. This failure of action was attributed to the limited solubility of Ast in aCSF (Brauns, O., Liepold, T., Radulovi'c, J., and Spiess, J. (2001) Neuropharmacology 41, 504-516). Studies which have been carried out in accordance with the present invention surprisingly revealed that the maximum solubility of [Glu¹¹]Ast (cmax=125 μM) in aCSF was found to be sixteen times higher than that of Ast (Table 4). Because the replacement of a hydrophobic residue by a Glu residue enhanced the solubility, we considered to increase the solubility even more by an additional Glu residue. Therefore, Leu¹⁶ of Ast was replaced by Glu corresponding to the Svg sequence (FIG. 1).The resulting acidic Ast analog [Glu^{11,16}]Ast (cmax=290 μM) showed a 40 times higher maximum solubility in aCSF compared with Ast (Table 4). However, [Glu¹¹]Ast which was much more soluble at physiological pH than Ast, appeared to be less charged than Ast based on IEF measurements. [Glu^{11,16}]Ast was bound by both CRFR subtypes with significantly higher affinity than Ast (IC₅₀=3.3 nM for rCRFR1 and IC50=1.1 nM for mCRFR2β). No specific binding of [Glu^{11,16}]Ast to rCRFBP was detectable. In the behavior experiments, OCRF was used because of its preference for CRFR1. It was observed that locomotion of mice was markedly decreased after icv injection of 20 pmol (90 ng) of oCRF as indicated by the total distance crossed within the tested time period (F2,21=3.0, p<0.05; FIG. 3). Similarly, anxiety-like behavior was enhanced as indicated by the reduced number of open arm visits (F2,21=2.6, p<0.05) and the percentage of time spent in the open arms of the maze (F2,21=2.7, p<0.05; FIG. 3). These behavior effects were significantly enhanced by the injection of 35 pmol (170 ng) of oCRF (F2,21=31.2, p<0.001; F2,21=17.7, p<0.001; F2,21=8.8, p<0.001; FIG. 4). When 60 pmol (230 ng) of [Glu¹¹]Ast was preinjected before the application of 20 pmol of oCRF, the oCRF-induced behavior changes were prevented (FIG. 3A). However, the dose of [Glu¹¹]Ast was not sufficient to generate a significant difference between the behavior responses elicited by oCRF alone and oCRF in the presence of antagonist. A higher concentration of [Glu¹¹]Ast could not be employed because of solubility limitations. The introduction of the second Glu residue to generate [Glu^{11,16}]Ast increased the solubility as mentioned above (Tab.4) and permitted the employment of higher doses. It was observed that the number of open arm visits and traveled distances of the mice treated with 20 pmol of oCRF and the mice treated with 20 pmol of OCRF and 120 pmol of [Glu^{11,16}]Ast differed significantly from one another (FIG. 3B). The same dose of [Glu^{11,16}]Ast injected prior to the application of 35 pmol of oCRF significantly attenuated the oCRF-induced behavioral effects in the plus-maze test (FIG. 3D). In contrast, no significant change of the oCRF-induced behavior effects was found when 120 pmol (450 ng) of α-hel-CRF 9-41 was preinjected before the application of 35 pmol of oCRF (FIG. 3C)

Thus, the introduction of the second Glu′ residue to generate [Glu^{11,16}]Ast increased the solubility as mentioned above (Tab.4) and permitted the employment of higher doses. It was observed that the number of open arm visits and traveled distances of the mice treated with 20 pmol of OCRF and the mice treated with 20 pmol of oCRF and of 120 pmol [Glu^{11,16}]Ast differed significantly from one another (FIG. 3B).The same dose of [Glu^{11,16}]Ast injected prior to the application of 35 pmol of oCRF significantly attenuated the oCRF-induced behavioral effects in the plus-maze test (FIG. 3D). In contrast, no significant change of the oCRF-induced behavior effects was found when 120 pmol (450 ng) of α-hel-CRF 9-41 was preinjected before the application of 35 pmol of OCRF (FIG. 3C)

Thus, it is envisaged that in another most preferred embodiment of the present invention amino acid Alanine at position 11 and amino acid Leucine at position 16 of the above depicted sequence of astressin are replaced with amino acid Glutamine.

In another preferred embodiment the present invention relates to an antagonist/agonist obtainable by the methods of the present invention. In a most preferred embodiment said antagonists of the invention are selected from a group consisting of [Glu¹¹Ast, [Glu^{11,16}]Ast. In another most preferred embodiment said agonists of the invention are selected from a group consisting of [A²¹]Svg, [A^{21,23}R²²]Svg and [E²²]h/rCRF.

In another embodiment of the present invention the antagonist/agonist of the present invention of is fused to another moiety, such as a heterologous protein, a label, a tag, an enzyme as indicated herein.

In a further embodiment the present invention relates to a polynucleotide encoding the agonist/antagonist of the present invention.

The polynucleotide of the present invention may be, e.g., DNA, cDNA, RNA or synthetically produced DNA or RNA or a recombinantly produced chimeric nucleic acid molecule comprising any of those polynucleotides either alone or in combination.

In another embodiment the present invention relates to a vector comprising the polynucleotide of the present invention.

The vector of the present invention may be, e.g., a plasmid, cosmid, virus, bacteriophage or another vector used conventionally in genetic engineering, and may comprise further genes such as marker genes which allow for the selection of said vector in a suitable host cell and under suitable conditions.

In a preferred embodiment of the vector of the present invention the polynucleotide is operatively linked to an expression control sequence.

Said expression control sequence allows expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions.

Possible regulatory elements permitting expression in prokaryotic host cells comprise, e.g., the PL, lac, trp or tac promoter in E. coli, and examples for regulatory elements permitting expression in eukaryotic host cells are the AOX1 or GALL promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogene), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences.

Furthermore, the present invention relates to a host comprising the polynucleotide or vector of the present invention.

Said host may be a prokaryotic or eukaryotic cell. The polynucleotide or vector of the invention which is present in the host cell may either be integrated into the genome of the host cell or it may be maintained extrachromosomally.

The host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a polynucleotide or vector of the present invention for the expression of the antagonist of the present invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the antagonist encoded by the polynucleotide of the present invention may or may not be post-translationally modified. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (e.g. Sambrook et al., Molecular Cloning: A Laboratory Manual, Cold Spring Harbor Laboratory, Cold Spring Harbor, N.Y., 1989). The genetic constructs and methods described therein can be utilized for expression of the antagonist of the present invention in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Furthermore, transgenic animals, preferably mammals, comprising host cells of the invention may be used for the large scale production of the antagonist of the present invention.

The present invention also relates to a method for producing the antagonist/agonist of the present invention, said method comprising culturing the host of the present invention under conditions that cause the synthesis of said agonist/antagonist, and recovering said antagonist from the culture.

Depending on the specific construct and condition used, the agonist/antagonist may be recovered from the host cells, from the culture medium or from both. Further, it is envisaged that the antagonist/agonist which was produced by the method of the invention is chemically modified afterwards as described herein (for example a lactam-bridge is incorporated into the astressin-derivatives of the invention).

The present invention further relates to an agonist/antagonist obtainable by the methods of the present invention.

Alternatively, the antagonist/agonist of the present invention can be chemically synthesized according to methods well known in the art, e.g., solid phase synthesis with Fmoc or t-boc chemistry (see also, e.g., Rühmann, A., A. K. E. Köpke, F. M. Dautzenberg, and J. Spiess, Proc. Natl. Acad. Sci. USA 93:10609-10613, 1996).

The invention further relates to a method of modifying an antagonist/agonist obtained by the methods of the invention as a lead compound to achieve (i) modified site of action, spectrum of activity, organ specificity, and/or (ii) decreased toxicity (improved therapeutic index), and/or (ill) decreased side effects, and/or (iv) modified onset of therapeutic action, duration of effect, and/or (v) modified pharmakinetic parameters (resorption, distribution, metabolism and excretion), and/or (vi) modified physico-chemical parameters (hygroscopicity, color, taste, odor, stability, state), and/or (vii) improved general specificity, organ/tissue specificity, and/or (viii) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiozolidines or combinations thereof.

The various steps recited above are generally known in the art. They include or rely on quantitative structure-action relationship (QSAR) analyses (Kubinyi, 1993), combinatorial biochemistry, classical chemistry and others (see, for example, Holzgrabe and Bechtold, 2000).

In a preferred embodiment the antagonists/agonists of the present invention are protected against peptidases by means and methods known in the art e.g. by incorporation of a D-amino acid residues for example the N-terminal D-Phe within Astressin, by acetylation e.g of the N-terminus of the peptidic antagonist/agonist of the invention and/or by coupling to a photoactivatable group.

Furthermore, the person skilled in the art is well aware that it is also possible to label the antagonists/agonists of the invention with an appropriate marker or tag for specific applications, such as for the detection of the presence of CRFBP and or the CRF-receptors in a sample derived from an organism, in particular mammals, preferably human. A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include radionuclides such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin, enzymes (like horse radish peroxidase, β-galactosidase, alkaline phosphatase), chemi- or bioluminescent compounds (like dioxetanes, luminol or acridiniums), fluorochromes (like fluorescein, rhodamine, Texas Red, etc.) or chromogenic agents as well as substrates, cofactors, inhibitors, magnetic particles and the like. Patents teaching the use of such labels include US Patents U.S. Pat. No. 3,817,837; U.S. Pat. No. 3,850,752; U.S. Pat. No. 3,939,350; U.S. Pat. No. 3,996,345; U.S. Pat. No. 4,227,437; U.S. Pat. No. 4,275,149 and U.S. Pat. No. 4,366,241. It is also envisaged that said tag is selected, but not limited to, from the group consisting of His-tag, Streptavidin-tag, HA-tag, GST-tag, CBP-tag, MBP-tag, FLAG-tag, myc as well as single-chain fragments (sc Fvs) of antibody binding regions. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art.

Labeling procedures, like covalent coupling of enzymes or biotinyl groups, iodinations, phosphorylations, biotinylations, are well known in the art. Detection methods comprise, but are not limited to, autoradiography, fluorescence microscopy, direct and indirect enzymatic reactions, FACS-analysis etc.

In another embodiment the present invention relates to an antibody directed against the agonist/antagonist of the present invention.

In a further embodiment the present invention relates to an anti-idiotypic antibody directed against the antibody of the present invention.

The antibodies of the present invention may be monoclonal antibodies, polyclonal antibodies, single chain antibodies, humanized antibodies, or fragments thereof that specifically bind the antagonist of the present invention or the antibody directed against the antagonist of the present invention. Bispecific antibodies, synthetic antibodies, antibody fragments, such as Fab, Fv or scFv fragments etc., or chemically modified derivatives of any of these are also encompassed by the present invention. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof can be obtained by using methods which are described, e.g., in Harlow and Lane “Antibodies, A Laboratory Manual”, CSH Press, Cold Spring Harbor, 1988. The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. As discussed above, the antibodies of the invention may exist in a variety of forms besides complete antibodies; including, for example, Fv, Fab and F(ab)2, as well as in single chains; see e.g. WO88/09344.

The present invention also relates to a pharmaceutical composition comprising the agonist/antagonist, the polynucleotide, the vector, the antibody and/or the anti-idiotypic antibody of the present invention and optionally a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

The present invention also relates to a diagnostic composition comprising the agonist/antagonist, the polynucleotide, the vector, the antibody and/or the anti-idiotypic antibody of the present invention.

Further, the present invention also relates to a kit comprising the agonist/antagonist, the polynucleotide, the vector, the antibody and/or the anti-idiotypic antibody of the present invention. The components of the diagnostic composition and/or the kit of the invention may be packaged in containers such as vials, optionally in buffers and/or solutions. If appropriate, one or more of said components may be packaged in one and the same container. Additionally or alternatively, one or more of said components may be adsorbed to solid support such as, e.g., a nitrocellulose filter or nylon membrane, or to the well of a microtiter plate.

Further, the present invention also relates to the use of the agonist/antagonist, the polynucleotide, the vector, the antibody and/or the anti-idiotypic antibody of the present invention for the preparation of a pharmaceutical composition for diagnosing, preventing and/or treating a corticotropin-releasing factor receptor-associated disease.

In a more preferred embodiment said corticotropin-releasing factor receptor-associated disease is affective disorders, gastric intestinal diseases, cardiopathic diseases, psychiatric diseases, preferably eating disorders, anxiety disorders or anorexia nervosa, and/or Alzheimer's disease.

In another embodiment the present invention relates to a method for preparing a pharmaceutical composition comprising carrying out a method of the invention; and formulating the obtained (i.e. improved) peptidic antagonist/agonist of CFRF into a pharmaceutical composition and, optionally, a pharmaceutically acceptable carrier and/or diluent.

The documents cited herein are herewith incorporated by reference.

Abbreviations used throughout the description, the figure legends, and the examples are as follows: IUPAC rules are used for the nomenclature of peptides including one letter codes for amino acids. AAA: amino acid analysis; ACTH: adrenocorticotropic hormone; ANOVA: one-way analysis of variance; astressin: {cyclo(30-33)[DPhe¹², Nle^{21,38}, Glu³⁰, Lys³³]h/rCRF₍₁₂₋₄₁₎}; BSA: bovine serum albumin; CAMP: adenosine 3′,5′-cyclic monophosphate; CRF: corticotropin-releasing factor (h=human, o=ovine, r=rat); CRFR: CRF receptor; DIEA: N,N-diisopropylethylamine; DMF: dimethylformamide; Fmoc: 9-fluorenylmethoxycarbonyl; HBTU: O-(benzotriazol-1-yl)-N,N,N′,N′-tetramethyluronium hexafluorophophate; HEK: human embryonic kidney; HOAc: acetic acid; HOBt: 1-hydroxybenzotriazole; ¹²⁵I: ¹²⁵I-iodinated; MeCN: acetonitrile; MS: mass spectrometry; NMP: N-methylpyrrolidone-2; OAll: O-allyl; OAlloc: O-allyloxycarbonyl; Pd⁰[PPh₃]₄: tetrakis-(tripheriylphosphine)-palladium; RP-HPLC: reverse phase high-performance liquid chromatography; SAR: structure-activity relationship; Svg: sauvagine; TFA: trifluoroacetic acid; Ucn: urocortin.

The figures show:

FIG. 1: Sequence comparison of CRF and Svg analogs. A dash marks an identical amino acid residue. The ARAE motif of h/rCRF and the aligned corresponding stretches of residues of other CRF-like peptides are boxed. The site of the Glu/Ala switch is underlayed in gray. The bracket between Glu³⁰ and Lys³³, of the Ast analogs depicts the lactam bridge connecting the side chains of Glu³⁰ and Lys³³. The following symbols are used: B=norleucine, Z=pyroglutamic acid, f=D-phenylaianine, ▪=C-terminal amide.

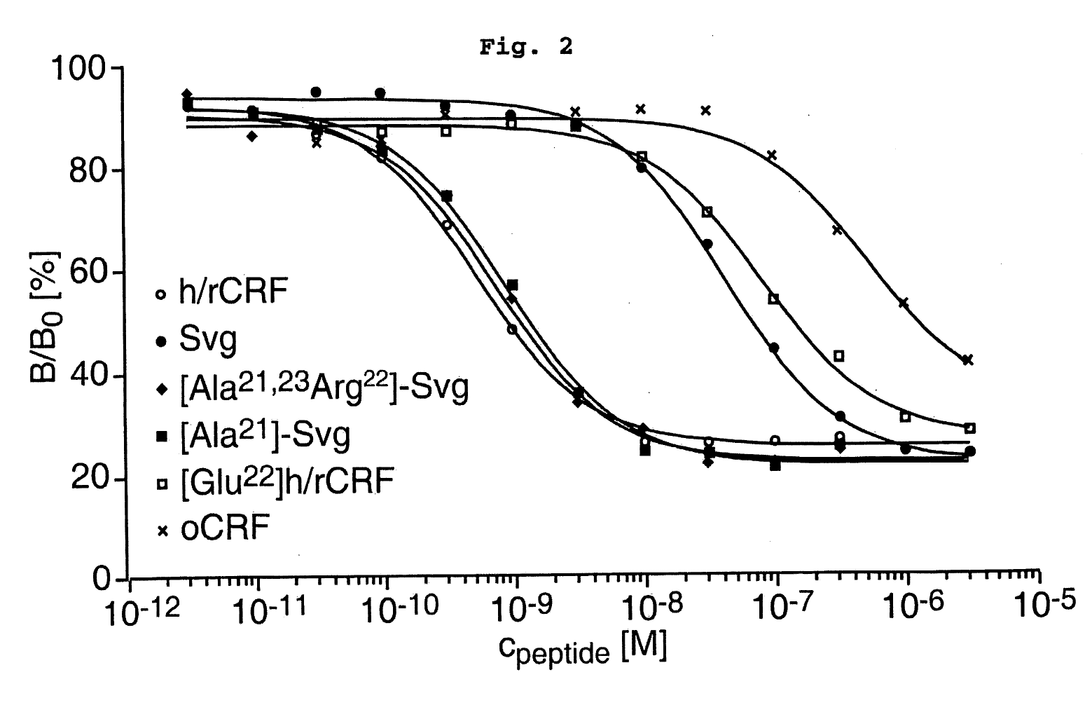

FIG. 2: Competitive binding of the CRF and Svg analogs to rCRFBP. Binding curves were normalized by total binding in the absence of competitor (B₀). Data points represent pooled data from at least four independent experiments.

FIG. 3: Potency of the antagonists [Glu¹¹]Ast and [Glu^{11,16}]Ast in the plus-maze behavior of C57BL/6J mice. The antagonists and agonists were injected icv 30 and 15 min, respectively, before exposure to the elevated plus-maze for 5 min. A, 60 pmol (230 ng) [Glu¹¹]Ast, 20 pmol (90 ng) OCRF; B, 120 pmol (430 ng) [Glu^{11,16}]Ast, 20 pmol oCRF; C, 120 pmol (450 ng) α-hel-CRF⁹⁻⁴¹, 35 pmol (170 ng) oCRF; D, 120 pmol [Glu^{11,16}]Ast, 35 pmol oCRF. Statistically significant differences were determined by t-tests: *, p<0.05 vs. ACSF; **p<0.01 vs. ACSF; ***, p<0.001 vs. ACSF; a, p<0.05 vs. OCRF; b, p<0.01 vs. OCRF; c, p<0.001 vs. OCRF.

FIG. 4: Helical wheel diagrams showing the internal amphiphilic helices of Svg and h/rCRF.

The examples illustrate the invention.

### EXAMPLE 1

### Production of rCRFBP rCRFR1, and mCRFR2β

rCRFBP was produced in HEK-293 cells stably transfected with cDNA coding for rCRFBP C-terminally fused with a His₆ sequence as described in Jahn et al (2001), Peptides 22, 47-56. rCRFR1 and mCRFR2β were obtained from membrane fractions prepared from HEK-293 cells stably transfected with cDNA coding for rCRFR1 and mCRFR2β respectively (Rühmann et al., Proc. Natl. Acad. Sci. (1998) 95, 15264-15269).

### EXAMPLE 2

### Binding and Competition Assays

Recombinant CRF binding protein was produced using human embryonic kidney (HEK) 193 cells stably transfected with cDNA encoding for rCRFBP C-terminally prolonged by a His₆-sequence.

Binding of CRF-like peptides to rCRFBP was analyzed using a scintillation proximity assay (SPA) developed in nickel chelate coated 96 well microtiter plates (Flash Plate PLUS, NEN). The competition assay consisted of 0.1 nM radiolabeled ligand [¹²⁵I-Tyr⁰]h/rCRF, unlabeled ligand (0 to maximal 3 μM), and medium containing His-tagged rCRFBP in a total volume of 200 μl PBS (pH 7.5)+0.02% (w/v) nonionic detergent NP-40. The sealed plates were incubated for 4 h at room temperature and then counted in a Microbeta scintillation counter (Wallac).

The SPA assay for rCRFR1 and rCRFR2β was performed in 96-well microtiter plates. The competition for rCRFR1 and rCRFR22 was carried out between of 0.05 nM radiolabeled ligands [¹²⁵I-Tyr⁰]h/rCRF and, [¹²⁵I-Tyr⁰]Svg, respectively, and unlabeled ligand (0 to maximal 3 μM) in a binding buffer containing 50 mM Tris, 5 mM MgCl₂, 100 KIU trasylol, 1 mM DTT, and 1% BSA. After 1 hour shaking 250 mg wheat germ agglutinin beads per well were added and the plates were counted in a microbeta scintillation counter (Wallac). cAMP accumulation was measured using the SPA based Biotrack assay (Amersham).

### EXAMPLE 3

### Alternative Competition Binding Assay

The utilization of a scintillation proximity assay (SPA) (Udenfriend, S., Gerber, L. D., Brink, L.&Spector, S.(1985)Proc.Nat. Acad.Sci.U.S.A.82, 8672-8676.) by using the lectin coated neuropeptide Y receptor SPA (Amersham Pharmacia) for CRFR binding analysis was established in our laboratory (Hofmann, B. A., Sydow, S., Jahn, O., van Werven, L., Liepold, T., Eckart, K. & Spiess, J. (2001) Prot. Sci. 10, in press.). Binding of CRF-like peptides to rCRFBP was carried out with an SPA assay in nickel-chelate-coated 96-well microtiter plates (Flash Plate PLUS™, NEN), which bound rCRFBP tagged with a C-terminal His6 sequence (rCRFBP-His6). The scintillator beads carrying the Ni 2+ ions for binding of the His tag were located at the inner surface of the wells of this microtiterplate. Unlabeled peptide (0 to maximal 3 μM), 0.1 nM radiolabeled ligand [125 I-Tyr 0]h/rCRF (NEN), and cell culture medium containing rCRFBP-His6 (Jahn, O., Eckart, K., Sydow, S., Hofmann, B. A. & Spiess, J. (2001) Peptides 22, 47-56) were mixed in a total volume of 200 μl assay buffer (PBS (pH 7.5) and 0.02%(w/v) nonionic detergent NP-40). The plates were sealed and incubated for four hours at room temperature. The radioactivities in the microtiterplates were counted in a Wallac 1450 Microbeta scintillation counter by detection of the light emitted from the scintillator beads. In both SPA systems the detection of the radioligand.8 depended on its proximity to the beads containing the scintillator. When the radioligand receptor complex was bound to one bead by specific interaction of CRFR or CRFBP with the lectin (neuropeptide Y receptor SPA) or the Ni 2+ ions (Flash Plate PLUS™), respectively, light was emitted from the scintillator on excitation by the radiation of the radioligand. The major portion of counted radioactivity (>70%) represented specific binding that was because of radiolabeled ligand specifically bound to CRFBP or CRFR. In the same manner, radioligand non-specifically bound to a bead could cause light emission which represented non-specific binding (<30%). Binding data were analyzed using the Prism computer program (GraphPad Software, San Diego).

### EXAMPLE 4

### Measurement of Intracellular CAMP Accumulation

The cells were stimulated as described (Sydow, S., Radulovic, J., Dautzenberg, F. M. & Spiess, J. (1997) Mol. Brain Res. 52, 182-193.) using increasing concentrations of h/rCRF or Svg. Intracellular CAMP was measured with the Biotrak™CAMP [¹²⁵I]SPA system (Amersham Pharmacia) according to the manufacturer's product manual.

### EXAMPLE 5

### Isoelectric Focusing

Isoelectric focusing (IEF) was carried out with a BioRad IEF cell system using Biorad IEF strips (11 cm, pH range from 3 to 10). The system was cooled to 20° C. Calibration was performed with 80 μg of Sigma IEF mix 3.6-9.3 dissolved in 200 μl water containing 0.2% Bio-Lyte® 3/10 and 0.1% NP40. Twenty five μg of each peptide was dissolved in 200 μl water containing 0.2% Bio-Lyte® 3/10,0.1% NP40, and 10 mM DTT.IEF gels were placed in the chamber containing the peptide solution. The IEF gels were initially rehydrated for 12 hours at 50 V. Focusing of the peptides was achieved by application of a linear voltage gradient starting at 250 V and reaching 8000 V in 2.5 hours. The gels were then exposed to 8000 V for 4.5 hours. Subsequently, the IEF gels were stained in the BioRad IEF gel staining solution and destained in a mixture of 10% acetic acid, 40% methanol, and 50% water.

### EXAMPLE 6

### Mass Spectrometry

To establish the hydrophobicity order of the peptidic CRF antagonists, peptide mixtures were chromatographed on a Vydac C18 column (150×0.3 mm) and passed through the UV cell connected to the ES interface of the mass spectrometer (AutoSpec-T, Micromass). A linear gradient of water and acetonitril (0.4% CH₃CN per min) with solutions containing 0.05-0.07% TFA was applied.

An on-line RP-HPLC-mass spectrometry (HPLC-MS) for the determination of the relative hydrophobicity of the CRF anatgonists is outlined in Jahn et al., Peptides (2001) 22, 47-56.

### EXAMPLE 7

### Synthesis of Astressin Derivatives

All peptides were synthesized using standard Fmoc chemistry and HBTU-activation as described recently (Rühmann et al., Proc. Natl. Acad. Sci. (1998) 95, 15264-15269). The peptides were characterized with a Biolon 20 plasma desorption mass spectrometer as outlined in Jahn et al., Peptides (2001) 22, 47-56.

The lactam bridge in Ast and [E²²]Ast was prepared as described recently (Rühmann et al., Proc. Natl. Acad. Sci. (1998) 95, 15264-15269).

### EXAMPLE 8

### Determination of the Maximum Solubility

Peptides were dissolved in 10 mM acetic acid and mixed with 2× concentrated aCSF. The concentration of each peptide in 10 mM acetic acid was adjusted so that a pellet was observed after mixing with the same volume of 2× concentrated aCSF. The final concentration in the supernatant was determined by amino acid analysis which was performed after hydrolysis of peptides (6 M HCl, 3 h, 9 150° C.) in the presence of norleucine as internal standard with a Beckman HPLC Analyzer System 6300 (Beckman Coulter, Fullerton, Calif.).

### EXAMPLE 9

### Elevated Plus Maze Test

C57BL/6J male mice, cannulated into the lateral brain ventricles, were injected with artificial cerebrospinal fluid (aCSF) or 220 ng [E¹¹]Ast. Ten min later the animals received 90 ng oCRF, and after 30 min, their behavior was recorded in the elevated plus maze test.

oCRF elicited anxious behavior of mice as demonstrated by the significantly decreased number of open arms visits, percentage of time spent in open arms of the maze, and the total distance crossed. These effects were fully abolished in mice preinjected with a threefold molar excess of [E¹¹]Ast. In vivo [E¹¹]Ast prevented anxious behavior in response to CRF. The good efficiency in the behavioral test was the result of the improved solubility of [E¹¹]Ast and the increased affinity of [E¹¹]Ast to both CRFR subtypes.

Alternatively, the elevated plus-maze behavior of C57BL/6J mice cannulated in the lateral ventricles (Radulovic, J., Rühmann, A., Liepold, T & Spiess, J. (1999) J.Neurosci. 19, 5016-5025) was investigated 15 min after injection of ovine CRF (oCRF) in the elevated plus-maze test (Radulovic, J., Kammermeier, J. & Spiess, J. (1998) Behav.Brain Res. 95, 179-189.).Peptide concentrations were determined by amino acid analysis (Radulovic, J., Rühmann, A., Liepold, T. & Spiess, J. (1999) J.Neurosci. 19, 5016-5025). CRF antagonists in aCSF as vehicle or vehicle alone were injected 30 min prior to oCRF administration. The behavior of the mice was recorded by a video camera connected to a PC and analyzed by the software VideoMot 2 (Technical &Scientific Equipment). The time spent, distance crossed, and number of entries in the open arms, closed arms, and center were recorded. The cannular placement was confirmed for each mouse by histological examination of the brains after methylene blue injection (Radulovic, J., Rühmann, A., Liepold, T. & Spiess, J. (1999) J.Neurosci. 19, 5016-5025). The behavioral data were analyzed by t tests or ANOVA followed by post hoc Scheffe's test for multiple comparisons. Data are presented as mean±SEM. The shift of preference from the open to the closed arms is interpreted as an increase of anxiety-like behavior. Locomotor activity is determined with this assay by the distance traveled.

### EXAMPLE 10

### Displacement Assay of CRF From rCRFBP

A displacement assay of CRF from rCRFBP can be carried out using a scintillation proximity assay (SPA) developed in nickel chelate coated 96 well microtiter plates (Flash Plate PLUS, NEN). The displacement assay would consist of 0.1 nM radiolabeled ligand [¹²⁵I-Tyr⁰]h/rCRF and medium containing His-tagged rCRFBP in a total volume of 150 μl of binding buffer (PBS (pH 7.5)+0.02% (w/v) nonionic detergent NP-40). After one hour incubation at room temperature, 50 μl binding buffer containing an unlabelled CRF-like peptide in a concentration of 4 μM or no peptide for control experiments would be added, the plates would be sealed and incubated for four at least 4 hours at 37° C. After incubation, the plates would be counted in a Microbeta scintillation counter (Wallac). The radioactivity difference between the wells containing the unlabelled peptide and the control wells represent the amount of radioactive peptide displaced from CRFBP.

### EXAMPLE 11

### Pharmacological Relevance of Residue 22 of h/rCRF

A new high throughput binding assay based on the scintillation proximity assay principle was developed for recombinant rCRFBP by employing the affinity of the His-tag of CRFBP to Ni 2+ ions. This assay did not require a separation of bound and free radioligand in contrast to the charcoal precipitation assay used earlier to characterize the pharmacological profile of rCRFBP. The SPA assay was validated through the known rank order of affinity of different CRF-like peptides as determined to be h/rCRF=α-hel-CRF⁹⁻⁴¹>>Svg>Ast>oCRF>>aSvg-30 (Table 1). This order was in agreement with results obtained with the charcoal precipitation assay (13). rCRFBP bound h/rCRF (IC50=0.54 nM) and [Ala 21,23 Arg 22]Svg (IC50=0.84 nM), which contained the amino acid residues of the ARAE motif of h/rCRF, with indistinguishable affinities in contrast to the lower affinity of Svg (IC50=57 nM, FIG. 2, Table 1). When the relative hydrophobicities and the conformational preferences (25) of the amino acid residues 22-24, Ala-Arg Ala, of h/rCRF and 21-23, Glu-Lys-Gln, of Svg were compared (Table 2), it was observed that all residues shared a high propensity for the formation of α-helical secondary structures. Major differences were found for residues Ala 24 of h/rCRF and Gin 23 of Svg which differed significantly in their relative hydrophobicity. Residues Ala 22 of h/rCRF and Glu 21 of Svg differed in their relative hydrophobicity and in the net charge of their side chains. Therefore, it was hypothesized that amino acid residue Ala 22 of h/rCRF and the corresponding residue Glu 21 of Svg were responsible for the affinity difference of these peptides to CRFBP. This hypothesis was tested by the synthesis of [Glu²²]h/rCRF and [Ala²¹]Svg (FIG. 1) and subsequent analysis of their binding to rCRFBP. The affinity of [Glu²²]h/rCRF (IC50=80 nM) was decreased by two orders of magnitude compared with h/rCRF. Similarly, the affinity of [Ala²¹]Svg (IC50=0.94 nM) compared with Svg was enhanced by two orders of magnitude (FIG. 2). The binding affinities of Svg and [Ala²¹]Svg to both CRFR subtypes did not deviate significantly from one another, whereas the binding affinity of [Glu²²]h/rCRF compared with that of h/rCRF was slightly enhanced (Table 1). However, the selectivity of h/rCRF favoring CRFR1 by a factor of “30 was not altered. A similar modification as described for h/rCRF was carried out for Ast. The acidic peptide [Glu¹¹]Ast (FIG. 1) obtained was analogous to [Glu²²]h/rCRF as Ast is to h/rCRF. As expected from the pharmacological profile of [Glu²²]h/rCRF, no detectable specific binding of [Glu¹¹]Ast to rCRFBP was found. The affinity of [Glu¹¹]Ast to both CRFR subtypes was increased by one order of magnitude compared with the affinity of Ast (Table 1). The relative intrinsic activity and potency of Ast and [Glu¹¹]Ast were obtained (Tab.3). To facilitate comparison of the most frequently used antagonists, α-hel-CRF⁹⁴⁻¹ and aSvg-30 were included in the study. The in vivo potency of the antagonists was assayed by intracerebroventricular (i.c.v.) injection of mice and subsequent behavioral analysis in the elevated plus-maze for anxiety-like behavior and locomotor activity, two behavioral responses modulated by CRF. It had been earlier observed that Ast preinjected i.c.v. did not prevent oCRF-induced changes of the mice's behavior in the elevated plus-maze. This failure of action was attributed to the limited solubility of Ast in aCSF. The maximum solubility of [Glu¹¹]Ast (cmax=125 μM) in aCSF was found to be sixteen times higher than that of Ast (Table 4). Because the replacement of a hydrophobic residue by a Glu residue enhanced the solubility, we considered to increase the solubility even more by an additional Glu residue. Therefore, Leu 16 of Ast was replaced by Glu corresponding to the Svg sequence (FIG. 1).The resulting acidic Ast analog [Glu¹¹,⁶]Ast (cmax=290 μM) showed a 40 times higher maximum solubility in aCSF compared with Ast (Table 4). However, [Glu 11]Ast which was much more soluble at physiological pH than Ast, appeared to be less charged than Ast based on IEF measurements. [Glu^{11,16}]Ast was bound by both CRFR subtypes with higher affinity than Ast (IC50=3.3 nM for rCRFR1 and IC50=1.1 nM for mCRFR2β). No specific binding of [Glu^{11,16}]Ast to rCRFBP was detectable. In the behavior experiments, oCRF was used because of its preference for CRFR1. It was observed that locomotion of mice was markedly decreased after icv injection of 20 pmol (90 ng) of oCRF as indicated by the total distance crossed within the tested time period (F2,21=3.0, p<0.05; FIG. 3). Similarly, anxiety-like behavior was enhanced as indicated by the reduced number of open arm visits (F2,21=2.6, p<0.05) and the percentage of time spent in the open arms of the maze (F2,21=2.7, p<0.05; FIG. 3). These behavior effects were significantly enhanced by the injection of 35 pmol (170 ng) of oCRF (F2,21=31.2, p<0.001; F2,21=17.7, p<0.001; F2,21=8.8, p<0.001; FIG. 4). When 60 pmol (230 ng) of [Glu¹¹]Ast was preinjected before the application of 20 pmol oCRF, the oCRF-induced behavior changes were prevented (FIG. 3A). However, the dose of [Glu¹¹]Ast was not sufficient to generate a significant difference between the behavior responses elicited by oCRF alone and oCRF in the presence of antagonist. A higher concentration of [Glu¹¹]Ast could not be employed because of solubility limitations. The introduction of the second Glu residue to generate [Glu^{11,16}]Ast increased the solubility as mentioned above (Tab.4) and permitted the employment of higher doses. It was observed that the number of open arm visits and traveled distances of the mice treated with 20 pmol of oCRF and the mice treated with 20 pmol of oCRF and of 120 pmol of [Glu^{11,16}]Ast differed significantly from one another (FIG. 3B). The same dose of [Glu^{11,16} ]Ast injected prior to the application of 35 pmol of oCRF significantly attenuated the oCRF-induced behavioral effects in the plus-maze test (FIG. 3D). In contrast, no significant change of the oCRF-induced behavior effects was found when 120 pmol (450 ng) of α-hel-CRF⁹⁻⁴¹ was preinjected before the application of 35 pmol of oCRF (FIG. 3C). The ligand requirements of CRFBP and CRFR are significantly different. It has been demonstrated that the central stretch of residues 6-33 of h/rCRF is sufficient for high-affinity binding to CRFBP, but not to CRFR. Svg and h/rCRF differ in this central part of 28 residues by 16 residues. Most importantly, the exchange of Ala 22 of h/rCRF by the corresponding Glu 21 of Svg switched off the high affinity binding to CRFBP without decreasing the affinity to CRFR. Consistently, the moderate affinity of Ast to rCRFBP was abolished by the introduction of Glu in the corresponding position. The acidic Ast analog [Glu^{11,16}]Ast, but also [Glu¹¹]Ast, (FIG. 1) did not show specific binding to rCRFBP, whereas the affinity to either CRFR subtype was increased (Table 1). By the introduction of the two Glu residues into the Ast.16 sequence to generate [Glu^{11,16}]Ast, the preference of Ast for rCRFR1 over rCRFBP was enhanced by a factor of more than 100. By using CD and NMR spectroscopic methods, evidence has been provided that CRF forms an amphiphilic helix whose hydrophobic patch binds to hydrophobic surfaces. On the basis of the helical wheel diagrams for the central parts of the peptides Svg and h/rCRF (FIG. 4), it is suggested that residues 21 and 22, respectively, were part of a hydrophobic patch composed of residues Ala 22, Leu. 15, Leu 8, Leu 19, and Phe 12 of h/rCRF (FIG. 4). It was demonstrated by CD that α-helical structures of h/rCRF and α-hel-CRF⁹⁻⁴¹ are involved in binding to CRFBP. In view of the crucial role of Ala 22 for binding to CRFBP, it is concluded that the hydrophobic patch may be important for binding to CRFBP. Consistently, Ala 24 located on the opposite site of the helical wheel of h/rCRF was found to be not important for binding to CRFBP. Interestingly, residues 8, 12, 15, and 19 of the hydrophobic patch are highly conserved throughout the CRF peptide family. A greater variability was found for residue 22 which is replaced by Ser, Thr, and Glu. The remarkable homology of the hydrophobic patch in urocortin and Svg, which are only homologous to an extent of 44% with h/rCRF, underlines its importance. On the basis of the importance of the hydrophobic patch of h/rCRF for high affinity-binding to CRFBP, it was speculated that the crucial contribution of Ala 22 in h/rCRF resulted from a direct hydrophobic interaction of the small Ala.17 side chain with the ligand-binding site of CRFBP. This interaction may have been disturbed by the charged bulky Glu residue in [Glu²²]h/rCRF as indicated by its lower affinity, whereas it may have been facilitated in [Ala²¹]Svg as demonstrated by the latter peptide's high affinity to CRFBP. This view is consistent with the observation that replacement of Thr 22 of oCRF by an Ala residue enhanced the affinity by one order of magnitude. The affinity difference of two orders of magnitude between Svg and h/rCRF depended mainly on the exchange of a single amino acid, of Glu for Ala or vice versa. From the results of IEF (Tab 4), it was concluded that Ast was positively charged in aCSF, whereas [Glu¹¹]Ast and [Glu^{11,16}]Ast carried a negative charge. In view of the observation that the isoelectric points of [Glu¹¹]Ast and [Glu^{11,16}]Ast were closer to the physiological pH than the isoelectric point of Ast, the solubility of these peptides obviously did not depend mainly on electric charges. It was more likely that it depended on an increasing hydrophilicity of the side chains which was detected even under the acidic conditions of reversed-phase HPLC-MS (Tab.4). [Glu^{11,16}]Ast was successfully used to prevent the oCRF-induced enhancement of anxiety-like behavior and decrease of locomotor activity of the mouse in the elevated plus-maze. The behavioral effects of [Glu^{11,16}]Ast were probably mediated by CRFR1 in view of the observations that oCRF binds preferentially to CRFR1 and that activation of CRFR1 in a novel environment results in reduction of locomotor activity. Alternatively, α-hel-CRF⁹⁻⁴¹ has been used for i.c.v. injection to inhibit CRFR1-mediated effects. Usually, doses of 260 pmol to 1.3 nmol (1 μg to 5 μg) of α-hel-CRF⁹⁻⁴¹ per mouse have been employed to inhibit CRFR1-mediated effects such as reduction of the locomotor activity in the elevated plus-maze. These reports are in agreement with our finding that 120 pmol of α-hel-CRF⁹⁻⁴¹ representing a threefold molar excess of the oCRF dose used did not prevent the oCRF-induced behavior changes. In contrast, a dose of 120 pmol (430 ng) of [Glu^{11,16}]Ast was sufficient to inhibit significantly the behavioral effects of oCRF in the elevated plus-maze. The increased in vivo potency of [Glu^{11,16}]Ast was facilitated by the markedly higher affinity to CRFR1 compared to Ast and α-hel-CRF9-41 in combination with the lack of detectable specific binding to CRFBP. Thereby, an absorption of the antagonist by binding to CRFBP and, in addition, release of endogenous CRF from CRFBP by the antagonist was prevented.

TABLE 1
Binding of various CRF-like agonists and antagonists to rCRFBP and CRF receptor
subtypes

| | | | IC₅₀ [nM] | | | |
|---|---|---|---|---|---|---|
| Peptide | rCRFBP | | rCRFR1 | | | mCRFR2β |
| h/rCRF | 0.54 | (0.38-0.71) | 1.6 | (1.3-1.9) | 42 | (25-59) |
| [Glu²²]h/rCRF | 80 | (70-90) | 0.61 | (0.44-0.79) | 16 | (15-18) |
| Svg | 57 | (45-70) | 0.52 | (0.29-0.74) | 0.92 | (0.72-1.1) |
| [Ala^{21,23}Arg²²]Svg | 0.84 | (0.66-1.0) | 0.29 | (0.06-0.51) | 0.82 | (0.56-1.1) |
| [Ala²¹]Svg | 0.94 | (0.60-1.3) | 0.32 | (0.15-0.49) | 1.1 | (0.89-1.3) |
| oCRF | 470 | (420-530) | 1.0 | (0.65-1.4) | 200 | (110-300) |
| Ast | 90 | (76-104) | 11 | (7.7-14) | 5.2 | (2.6-7.8) |
| [Glu¹¹]Ast | n.b. | | 1.4 | (0.94-1.9) | 0.58 | (0.54-0.63) |
| [Glu^{11,16}]Ast | n.b. | | 3.3 | (2.8-3.7) | 1.1 | (0.87-1.3) |
| aSvg-30 | n.b. | | 370 | (330-400) | 0.30 | (0.24-1.36) |
| α-hel-CRF⁹⁻⁴¹ | 1.0 | (0.9-1.0) | 61 | (52-69) | 4.3 | (3.3-5.3) |

IC₅₀ values are the mean of at least four experiments performed in duplicate. 95% confidence intervals are given in parentheses; n.b.: no specific binding with up to 3 μM inhibitor

TABLE 2
Relative hydrophobicities and conformational preferences of the differing amino acids
of the ARAE motif in h/rCRF and the corresponding stretch in Svg

| | | Secondary | | |
|---|---|---|---|---|
| | | structure propensities | | |
| | | α-helix | β-structure | turn structure |
| Equivalent residue | Rel. hydrophobicity | (P_{α}) | (P_{β}) | (Pₜ) |
| h/rCRF/Svg | h/rCRF/Svg | h/rCRF/Svg | h/rCRF/Svg | h/rCRF/Svg |
| Ala²²/Glu²¹ | 0.25/−0.62 | 1.41/1.59 | 0.72/0.52 | 0.82/1.01 |
| Arg²³/Lys²² | −1.8/−1.1 | 1.21/1.23 | 0.84/0.69 | 0.90/1.07 |
| Ala²⁴/Gln²³ | 0.25/−0.69 | 1.41/1.27 | 0.72/0.98 | 0.82/0.84 |

The relative hydrophobicities were taken from the consensus hydrophobicity scale of Eisenberg (24). P_{α, P}_{β, and P}ₜ represent the normalized frequencies for each conformation (25)

TABLE 3
Relative intrinsic activities and relative potencies of various CRF-like antagonists

| | Rel. intrinsic activity^{a} [%] | | Rel. potency of antagonist [%] | |
|---|---|---|---|---|
| Antagonist | rCRFR1 | mCRFR2β | rCRFR1 | mCRFR2β |
| Ast | 9.8 (9.3-10.3) | 1.1 (0.9-1.2) | 86 (85-87) | 99 (99-98) |
| [Glu¹¹]Ast | 8.4 (7.9-8.8) | 0.84 (0.67-1.0) | 86 (85-86) | 98 (99-98) |
| [Glu^{11,16}]Ast | 9.1 (8.4-9.4) | 1.0 (0.76-1.3) | 83 (82-85) | 97 (96-97) |
| aSvg-30 | 6.5 (6.1-6.8) | 1.0 (0.9-1.1) | 2.0 (0-6.9) | 99 (98-99) |
| α-hel-CRF⁹⁻⁴¹ | 19 (18-20) | 1.4 (1.2-1.5) | 14 (13-15) | 97 (96-97) |

^{a}The intrinsic activity was determined by the stimulation of rCRPR1-HEK 293 cells
and mCRFR2β-HEK 293 cells with 10 pM h/rCRF and Svg, respectively
$\begin{matrix}Rel . intrinsic activity [ \% ] : \\ \frac{cAMP ( 1 μM antagonist )}{cAMP ( 10 nM agonist )} \times 100\end{matrix}$
$\begin{matrix}Rel . potency [ \% ] : \\ 100 - \frac{cAMP ( 1 nM agonist + 1 μM antagonist )}{cAMP ( 1 nM agonist )} \times 100\end{matrix}$

TABLE 4
RP-HPLC retention time, isoelectric point, and
maximum solubility (cₘₐₓ) in aCSF of CRF agonists and
antagonists

| | Retention | | |
|---|---|---|---|
| | time¹ | Isoelectric | cₘₐₓ |
| Peptide | t [min] | point² | [μM] |
| Svg | 39:12 | 5.1 | n.d.³ |
| [Glu²²]h/rCRF | 41:41 | 5.6 | n.d.³ |
| [Ala²¹]Svg | 42:32 | 7.4 | n.d.³ |
| oCRF | 42:51 | 6.4 | n.d.³ |
| h/rCRF | 47:51 | 5.9 | n.d.³ |
| aSvg-30 | 19:51 | >9.5 | >4000⁴ |
| [Glu^{11,16}]Ast | 29:24 | 6.2 | 290 |
| [Glu¹¹]Ast | 30:25 | 7.0 | 120 |
| Ast | 35:17 | 8.9 | 8 |
| α-hel-CRF⁹⁻⁴¹ | 42:41 | 5.3 | 1480⁴ |

¹retention time for the antagonists and the agonists were determined in separate HPLC runs and can not be compared
²isoelectric points were determined by isoelectric focussing
³n.d = not determined
⁴Brauns et al. (16)

1. A method for improving the antagonistic/agonistic properties of peptidic antagonists/agonists of the corticotropin-releasing factor receptor (CRFR) comprising the steps of
(a) aligning the amino acid sequences of at least two antagonists/agonists of the corticotropin-releasing factor receptor (CRFR) which differ in their antagonistic/agonistic properties;
(b) identifying at least one position wherein the amino acid sequences are different;
(c) exchanging or replacing at least one amino acid which is different in the aligned amino acid sequences; and
(d) comparing the difference in the antagonistic/agonistic properties of the antagonists/agonists which comprise at least one exchanged or replaced amino acid and thereby identifying at least one amino acid which is responsible for said difference.

2. The method of claim 1 further comprising a step of replacing the amino acid identified in step (d) in a further peptidic antagonist/agonist of the corticotropin-releasing factor receptor (CRFR).

3. The method of claim 1 or 2 further comprising the step of refining the obtained antagonist/agonist by means of peptidomimetics.

4. The method of any one of claims 1 to 3, wherein said peptidic antagonist/agonist is selected from the group consisting of CRF, sauvagine, urotensin 1, urocortin and urocortin like peptide.

5. The method of claim 4, wherein said antagonist is astressin.

6. An antagonist/agonist obtainable by the method of any one of claims 1 to 5.

7. An astressin-derivative comprising or alternatively consisting of the amino acid sequence Phe¹-His²-Leu³-Leu⁴-Arg⁵-Glu⁶-Val⁷-Leu⁸-Glu⁹-norleucin¹⁰-Ala¹¹-Arg¹²-Ala¹³-Glu¹⁴-Gln¹⁵-Leu¹⁶-Ala¹⁷-Gln¹⁸-Glu¹⁹-Ala²⁰-His²¹-Lys²²-Asn²³-Arg²⁴-Lys²⁵-Leu²⁶-norleucin²⁷-Glu²⁸-Ile²⁹-Ile³⁰-NH₂, wherein
(a) Glu at position 19 and Lys at position 22 are connected via a lactam-bridge; and
(b) at least Ala at position 11 is replaced by an amino acid selected from the group consisting of an acidic amino acid and/or a charged amino acid.

8. The derivative of astressin of claim 7 wherein amino acid Leu at position 16 is replaced by an amino acid selected from the group consisting of an acidic amino acid and/or a charged amino acid.

9. The derivative of astressin of claims 7 or 8 wherein Ala at position 11 and Leu at position 16 is replaced with Glu.

10. An antagonist selected from the group consisting of [Glu¹¹]Ast and [Glu^{11,16}]Ast.

11. An agonist selected from the group consisting of [A²¹]Svg, [A^{21,23}R²²]Svg and [E²²]h/rCRF.

12. The derivative of astressin of any one of claims 7 to 9, the antagonist of claim 10, the agonist of claim 11 or the antagonist/agonist obtainable by the methods of any one of claims 1 to 5 which is fused to a heterologous polypeptide.

13. The derivative of astressin of any one of claims 7 to 9, the antagonist of claim 10, the agonist of claim 11 or the antagonist/agonist obtainable by the methods of any one of claims 1 to 5 which is modified and/or labeled.

14. A pharmaceutical composition comprising a derivative of astressin of any one of claims 7 to 9, 12 or 13, the antagonist of claim 10, the agonist of claim 11 or the antagonist/agonist obtainable by the methods of any one of claims 1 to

15. A diagnostic composition comprising a derivative of astressin of any one of claims 7 to 9, 12 or 13, the antagonist of claim 10, the agonist of claim 11 or the antagonist/agonist obtainable by the methods of any one of claims 1 to 5.

16. A kit comprising comprising a derivative of astressin of any one of claims 7 to 9, 12 or 13, the antagonist of claim 10, the agonist of claim 11 or the antagonist/agonist obtainable by the methods of any one of claims 1 to 5.

17. Use of the derivative of astressin of any one of claims 7 to 9, 12 or 13, the antagonist of claim 10, the agonist of claim 11 or the antagonist/agonist obtainable by the methods of any one of claims 1 to 5 for the preparation of a pharmaceutical composition for diagnosing, preventing and/or treating a Corticotropin-releasing factor receptor-associated disease.

18. The use of claim 17 wherein said Corticotropin-releasing factor receptor-associated disease is affective disorders, gastric intestinal diseases, cardiopathic diseases, psychiatric diseases, preferably eating disorders, anxiety disorders or anorexia nervosa, and/or Alzheimer's disease.

19. A method for preparing a pharmaceutical composition comprising
a) carrying out a method of any one of claims 1 to 5; and
b) formulating the obtained antagonist/agonist into a pharmaceutical composition and, optionally, a pharmaceutically acceptable carrier and/or diluent.


SEQUENCE LISTING (9 sequences)

SEQ ID NO: 1 - protein, 40 aa, Phyllomedusa sauvagei
  PEPTIDE (1)..(40)
EGPPISIDLSLELLRKMIEIEKQEKEKQQAANNRLLLDTI

SEQ ID NO: 2 - protein, 40 aa, Phyllomedusa sauvagei
  MOD_RES (1)..(1): PYRROLIDONE CARBOXYLIC ACID
EGPPISIDLSLELLRKMIEIAKQEKEKQQAANNRLLLDTI

SEQ ID NO: 3 - protein, 41 aa, Ovis aries
  MOD_RES (41)..(41): AMIDATION
SQEPPISLDLTFHLLREVLEMTKADQLAQQAHSNRKLLDIA

SEQ ID NO: 4 - protein, 41 aa, derived from Homo sapiens
  MOD_RES (41)..(41): AMIDATION
SEEPPISLDLTFHLLREVLEMERAEQLAQQAHSNRKLMEII

SEQ ID NO: 5 - protein, 30 aa, derived from Phyllomedusa sauvagei
  MOD_RES (30)..(30): AMIDATION
FHLLRKMIEIEKQEKEKQQAANNRLLLDTI

SEQ ID NO: 6 - protein, 30 aa, derived from Homo sapiens; identical to SEQ ID NO: 5
  MOD_RES (1)..(1): d-phenylalanine

SEQ ID NO: 7 - protein, 30 aa, derived from Homo sapiens
  MOD_RES (30)..(30): AMIDATION
FHLLREVLEBERAEQLAQEAHKNRKLBEII

SEQ ID NO: 8 - protein, 30 aa, derived from Homo sapiens
  MOD_RES (30)..(30): AMIDATION
FHLLREVLEBERAEQEAQEAHKNRKLBEII

SEQ ID NO: 9 - protein, 41 aa, Homo sapiens
  MOD_RES (1)..(1): AMIDATION
SEEPPISLDLTFHLLREVLEMARAEQLAQQAHSNRKLMEII